(19) United States
(12) Patent Application Publication
(10) Pub. No.: US 2012/0190113 A1
(43) Pub. Date: Jul. 26, 2012

(54) Macroporous Microcarrier Specific to Liver Cell, Preparation Method and Use Thereof

(76) Inventors: Yi Gao, Guanghzou (CN); Mingxin Pan, Guangzhou (CN); Duhui Gong, Guangzhou (CN); Zhi Zhang, Guangzhou (CN); Huancheng Zhou, Guangzhou (CN); Zhiwei Hu, Guangzhou (CN)

(21) Appl. No.: 13/389,595

(22) PCT Filed: Apr. 17, 2010
(86) PCT No.: PCT/CN2010/071860

§ 371 Date: Apr. 11, 2012

(30) Foreign Application Priority Data
Aug. 11, 2009 (CN) 2009 10041767.8
Aug. 11, 2009 (CN) 2009 10041770.X

(51) Int. Cl. C12N 5/071

(52) U.S. Cl. 435/395

(57) ABSTRACT

The present invention provides a macroporous microcarrier specific to hepatocytes using silk fibroin and galactosylated chitosan as main raw material, a preparation method thereof, and application for hepatocyte culture under the culture condition of microgravity rotation. The macroporous microcarrier s a sphere prepared from silk fibroin and galactosylated chitosan under the effect of crosslinker, wherein based on the total weight of the sphere, the content of silk fibroin is 50-80 wt % and the content of galactosylated chitosan is 15-40 wt %. The diameter of the microcarrier is 200-500 μm, and the aperture of the microcarrier is 40-80 μm. Compared with normal solid scaffold material, the microcarrier provided by the present invention has larger surface area/volume ratio and, a sinus gap structure extremely similar with in-vivo liver sinus structure, therefore it is more conducive to adhering of the hepatocytes on the scaffold material, contacting between cells, transporting oxygen and nutrient components and excreting metabolic products.

### TECHNICAL FIELD

The present invention relates to macroporous microcarrier for cell culture, and the preparation method and application thereof. Especially, the present invention relates to macroporous microcarrier specific to hepatocytes using silk fibroin and galactosylated chitosan as main raw material, and the preparation method and application thereof

### BACKGROUND OF THE INVENTION

Bioartificial liver is an extracorporeal artificial liver support system which has been developed since the mid- or late-1980's, and the bioreactor, as the hardcore of bioartificial liver, is constituted by hepatocytes and biosynthetic material. How to constitute a bioreactor culture system with high quality, abundant quantity and strong security is a most important and difficult technical problem in the field of bioartificial liver research. Hepatocytes are anchorage-dependent cells with polarity, which require an insoluble extracellular matrix for survival, recombinant proliferation and functioning. Intracorporeal hepatocytes exist in a three-dimensional, environment, and interaction between cells will help to adjust the growth and functional differentiation of cells. If a similar intracorporeal three-dimensional environment could be provided for hepatocytes in extracorporeal culture, the growth and function maintaining of hepatocytes would be improved. Therefore, it is possible to solve such problems in hepatocyte tissue culture by preparing a scaffold with three-dimensional stephanoporate reticular structure to simulate the intracorporeal environment. Macroporous microcarrier has full communicating sulcus, so that it can furthest increase the specific surface area Because the cells are fixed in the holes for growth sufficient growth space and larger adhesion area could be provided for the cells, also it benefits absorbing nutrient components and excreting metabolic products, so as to prevent the inhibition effect on cell growth, and increase the cell culture density and metabolism activity. Compared with other carriers, macroporous microcarrier has a series of advantages, for example: larger specific surface area, which is several times or even dozens of times larger than that of solid microcarrier; security of the cells growing in the holes from shearing damnification; three-dimensional cell growth, the cell density of which is more than 10 times of the solid microcarrier, possibly 10⁸/ml; high concentration of macroporous microcarrier, which would increase surface collision to promote the cell growth in holes, on the contrary, a concentration of solid microcarrier over a certain degree leading to lower cell density; suitable for long-term cell culture with good growth condition; suitable for protein producing and product excreting. However, existent macroporous microcarriers used in hepatocyte culture are not specific to hepatocytes, it is not capable to induce and improve the adhesion performance of hepatocytes on such microcarriers, and the cells are easy to detach during scale-up culture. Therefore, it is significant to find an ideal macroporous microcarrier specific to hepatocytes for large-scale extracorporeal hepatocyte culture.

### SUMMARY OF THE INVENTION

One of the purposes of the present invention is to provide a new macroporous microcarrier, which has hepatocyte affinity, benefits cell adhesion and proliferation, is capable to support plentiful cell growth and to maintain cell activity and functions in low cost, and is suitable for large-scale hepatocyte culture.

Another purpose of the present invention is to provide a preparation method for said macroporous microcarrier. The raw material used in this method is easy to obtain with low price, and the process of this method is simple.

Another purpose of the present invention is to provide a method for large-scale extracorporeal hepatocyte culture. This method combines said new microcarrier with microgravity rotary culture system, to achieve hepatocytes with strong functions and high density.

To achieve such purposes, one aspect of the present invention is to provide a macroporous microcarrier which has high hepatocyte affinity. Said macroporous microcarrier is a sphere prepared from silk fibroin and galactosylated chitosan, under the effect of crosslinker, wherein based on the total weight of the sphere, the content of silk fibroin is 50-80 wt % and the content of galactosylated chitosan is 15-40 wt %. In the present invention, the content of silk fibroin and the content of galactosylated chitosan, are mensurated by determining the concentration of silk fibroin and the concentration of galactosylated chitosan respectively, and then mixing together according to a desired volume counted by a preconcerted ratio. As shown under scanning electron microscope, the diameter of said microcarrier is 200-500 μm, the aperture of said microcarrier is 40-80 μm, and the porosity of said microcarrier reaches 95%. In one embodiment of the present invention, said macroporous microcarrier could be prepared by the following method, which comprises steps of:
A. mixing silk fibroin solution and galactosylated chitosan solution according to a ratio to obtain a SF/GC (silk fibroin-galactosylated chitosan) mixed solution with a final concentration of 4-7 w/v %;
B. dropping said SF/GC mixed solution into an oil phase of emulsifier under stirring, to obtain a white emulsion; dropping crosslinker into said white emulsion slowly, and stirring until aqueous phase is crosslinking solidified;
C. adding said white emulsion obtained by step B into a polar solvent with a pH value of 9-10 under stirring, keepping stirring for 40-60 minutes, and then filtrating to obtain microspheres which do not stick to each other;
D. solidifying said microspheres, removing surface oil phase, and sieving to obtain microspheres with a diameter of 200-500 μm; and
E. removing the crosslinker residual in said microspheres, and freeze drying to obtain said macroporous microcarrier.

Preferably, said emulsifier is Span 80: said oil phase is liquid paraffin; said crosslinker is glutaraldehyde; said polar solvent is isopropanol, ethanol and/or acetone.

In another embodiment of the present invention, said step E comprises a process of immersing said microspheres in a sucrose solution of high concentration before freeze drying.

In another embodiment of the present invention, said preparation method for macroporous microcarrier comprises a process of sterilization after step E. Preferably, said sterilization process is irradiating with cobalt 60-γ radial or autoclaving in distilled water or PBS solution. Another aspect of the present invention is to provide a method for large-scale extracorporeal hepatocyte culture, which comprises steps of:

I. providing said macroporous microcarriers; and

II. applying said macroporous microcarriers to microgravity rotary culture system.

In one embodiment of the present invention, said method for large-scale extracorporeal hepatocyte culture comprises a process of immersing said macroporous microcarriers in a phosphate buffer solution without calcium and magnesium (PBS) which has a concentration of 0.1 mol/L and a pH value of 7.0 over night, and then immersing in serum-containing culture medium for at least 10 hours before step II.

In the embodiment of the present invention, concentrated static inoculation method is used in said microgravity rotary culture system for cell inoculation. Regarding said concentrated static inoculation method, the cell inoculation concentration is preferable 2×10⁵/ml to 1×10⁶/ml; the initial medium volume is preferable 40%˜90% of the volume of the culture flask; the static time is preferable 12˜24 hours; and the initial rotary speed is preferable 7.6˜9 rmp.

In a preferable embodiment, the serum concentration of the culture medium used in said concentrated static inoculation method is 10%˜15%; and the culture medium is DMEN or PRMI 1640. Preferably, the culture medium contains. HEPESwith a concentration of 20 mmol/L˜50 mmol/L.

An advantage of the present invention is using a new SF/GC macroporous microcarrier having hepatocyte specificity for hepatocyte culture under simulative microgravity rotary culture condition, to build a simple and reliable extracorporeal hepatocyte culture method which has high cell density and high differentiation scale. This method is suitable for high density hepatocyte culture, because said SF/GC, macroporous microcarrier has a sinus gap structure extremely similar with the in-vivo liver sinus structure, it has specific hepatocyte adsorption effect, it can provide a wide growing space for hepatocytes, and it can achieve an efficient communication for nutrition, oxygen and metabolic products between hepatocytes and the medium, thus it realizes a high-density extracorporeal culture, reaching 10⁷/ml. Also, this method can provide a durative simulative microgravity environment in microgravity rotary culture, which helps hepatocyte proliferation, differentiation and intercellular contaction, and a three-dimensional structural tissue would be formed to improve hepatocyte functions in extracorporeal culture. This method could reduce damage on hepatocytes, because membrane oxygen and gas exchange is used in the microgravity rotary culture, no air bubbles and eddy would be produced, which would reduce the shearing force. Further, this method improves the cell inoculation efficiency as known that hepatocytes have high oxygen consumption, especially during the initial adherent stage of the culture, and they are greatly sensitive to shearing force. The relative concentration of the cells and microcarriers would be increased by concentrating the cells and microcarriers suspension in inoculation stage, and the contact opportunity between the cells and microcarriers would be increased, also a gas-liquid plane would be produced in the rotary culture flask to reduce the distance between the cells and the gas-liquid plane, and efficient gas exchange with the environment (i.e. incubator) would be achieved by turn on the valve or the flask cap. Therefore this method is more conducive to adhering of the hepatocytes on the scaffold material, contacting between cells, transporting oxygen and nutrient components and excreting metabolic products, so as to further improve the culture density of hepatocytes in vitro and the functions of hepatocytes.

### BRIEF DESCRIPTION OF THE DRAWINGS

FIG. 1 is a ¹HNMR spectrum of the macroporous microcarriers of the present invention.

FIG. 2 is a FITR spectrum of the macroporous microcarriers of the present invention.

FIG. 3 is a morphological image (40×) of the macroporous microcarriers of the present invention under inverted microscope.

FIGS. 4a and 4b are SEM images of the macroporous microcarriers of the present invention.

FIG. 5 is a SEM image of the interior structure of the macroporous microcarriers of the present invention.

FIG. 6 is an image of CL-1 cells with SF/GC macroporous microcarrier observed under inverted microscope at the sixth day of the culture.

FIG. 7 is an image of CL-1 cells with SF/GC macroporous microcarrier observed under scanning electron microscope (SEM) at the eighth day of the culture.

FIG. 8 is an enlarged image of CL-1 cells with SF/GC macroporous microcarrier observed under scanning electron microscope (SEM) at the eighth day of the culture.

FIG. 9a is an image of CL-1 cells observed under electron microscope at the sixth day of static culture using SF/GC macroporous microcarriers of the present invention.

FIG. 9b is an image of CL-1 cells observed under electron microscope at the sixth day of microgravity culture using SF/GC macroporous microcarriers of the present invention.

FIG. 10 shows the urea concentration in supernate of CL-1 cells cultured on SF/GC macroporous microcarriers under static or microgravity condition.

FIG. 11a is an image of CL-1 cells observed under electron microscope at the sixth day of microgravity culture using solid microcarriers (cytodex).

FIG. 11b is an image of CL-1 cells observed under electron microscope at the sixth day of microgravity culture using the macroporous microcarriers (SF/GC) of the present invention.

FIG. 12 shows a comparison of the urea concentration in supernate of CL-1 cells cultured on the microcarriers shown in FIGS. 11a and 11b under microgravity

FIG. 13a is an image of CL-1 cells observed under electron microscope at the sixth day of microgravity culture using porous microcarriers (cytodex).

FIG. 13b is an image of CL-1 cells observed under electron microscope at the sixth day of microgravity culture using the macroporous microcarriers (SF/GC) of the present invention.

FIG. 14 shows a comparison of the urea concentration in supernate of CL-1 cells cultured on the microcarriers shown in FIGS. 13a and 13b under microgravity.

### DETAILED DESCRIPTION OF THE PREFERRED EMBODIMENTS

Foresaid and/or further aspects and advantages of the present invention would be described in following embodiments accompany with experiment evidence, to make them clearer and easier to understand. Some nomenclatures should be defined and clarified before description of the embodiments and experiments.

Silk fibroin is a natural strutural protein without physiological activity. Silk fibroin is mainly constituted by three simple amino acids, glycine, alanine and serine, which account for 85% of the total amount of the protein. The recent researches indicate that silk fibroin has great biocompatibility, no toxicity, no irritativeness and has a characteristic of promoting cell adhesion and proliferation, therefore it has increasing extensive applications in biomedical fields. The applications in cell culture matrices or modification on biopolymers to improve their biological performance for tissue engineering are hotspots of the recent researches on silk fibroin.

Chitosan is a natural biopolymer that has been widely used in biomedicine and tissue engineering. Chitosan not only has biofunctions and the characteristics of biocompatibility, low toxicity and nearly non-irritability but also has high chemical reactivity. Thus it is easy to be modified by chemical reagents to improve its performance. Galactosyl is a ligand specific to asialoglycoprotein receptor on the surface of hepatocyte, and it can induce and improve hepatocytes adhering on extracellular matrix scaffold materials. Galactosylated chitosan is prepared by introducing galactosyl into the structure of chitosan under the activation of EDC and NHS, so as to modify the surface characteristic of chitosan. Further, SE/GC macroporous microcarrier is prepared by composing, silk fibroin and galactosylated chitosan. SF/GC macroporous microcarrier can provide a larger three-dimensional growing space for extracorporeal hepatocyte culture, also it can obviously improve the hepatocyte functions and the cell viability, therefore it is ideal porous microcarrier specific to hepatocyte.

Bioreactor is a bioartificial liver which takes a crucial part in the curative effect of the therapy for liver function failure. Theoretically, bioreactor extremely simulates the tissue structure of the normal liver, and provides a similar intracorporeal survival and metabolism environment for hepatocyte culture. Therefore an ideal bioreactor should have following features: a cell density of 1×10⁷ cells/mL, volumizing capability according to requirement and a volume up to several liters; continuous perfusion culture, achieving bidirectional substance transfer for nutrient components, oxygen and metabolic products; automatic online detection and adjustment of cell states, medium pH value and oxygen concentration etc., to make it convenient for medical workers to monitor and operate; hepatocyte metabolism function at least at the level of monolayer culture and at least lasting for 2 weeks; convenience for transportation and assembly. Now commonly used bioreactors include hollow fiber bioreactor, roller bottle culture reactor, stirred culture reactor, airlift culture reactor and microgravity rotary culture reactor etc., wherein hollow fiber bioreactor has been widest used because of the capability of isolating foreign proteins and preventing the human prexisting antibodies specific to antigens on foreign cells from killing the loaded cells, therefore it is quite suitable for foreign cell (such as pig hepatocyte) culture. However this kind of bioreactor has several defects; limited volume and low cell loadage, limited exchange area between medium and hepatocytes, which is not good for hepatocyte function maintaining and proliferation; easy blocking in side holes of semipermeable membrane by cell mass, which would affect exchange efficiency; unsuitability for large-scale freeze store, because hollow fiber can not tolerate deep low temperature. Other kinds of reactors, such, as roller bottle culture reactor and stirred culture reactor have a defect of large shearing force, thus they can not carry out high efficient and uniform cell inoculation and transportation for oxygen, nutrient components and cellular metabolic products, and thus achieve large-scale cell culture with high density and large volume, and long-term maintaining of cell functions. According to the relevant research in the past 20 years, compared with other tissue engineering bioreactors, rotary cell culture system (RCCS), which was initially designed by American Aeronautics and Space Administration (NASA) and has been applied to microgravity bioscience fields, has advantages of durative simulative microgravity environment which helps cell proliferation and differentiation, helps intercellular contaction and helps forming a three-dimensional structural tissue; membrane oxygen and gas exchange without bringing air bubbles and eddy; and low shearing force, thus mechanical damage on cells could be reduced. RCCS has wide application prospects, and has been successfully and widely used in many tissue engineering fields, such as rabbit corneal cells, skeletal muscle cells, osteoblasts, embryonic stein cells, cortical neurons and adipose tissue.

One aspect of the present invention is to provide a new macroporous microcarrier, which can be used in large-scale extracorporeal hepatocyte culture. Another aspect of the present invention is to provide a method for large-scale extracorporeal hepatocyte culture by using said macroporous microcarrier, Silk fibroin and galactosylated chitosan used in embodiments could be bought in the market, or be prepared by following methods. All reagents materials and apparatus used in embodiments could be bought in the market. Abbreviations and appellations used in the description have the meanings known to those skilled in this technical field.

Preparation for Macroporous Microcarrier

### The First Embodiment

A. Silk fibroin preparation: Add 75 g raw silk to a sodium carbonate solution with a concentration of 5 g/L and a volume of 2 L, boil for 0.5 hour and repeat twice; wash with abundant distilled water to remove sericin protein and dry at 60-70 ° C. to obtain silk fibroin; dissolve appropriate amount of silk fibroin in a calcium chloride/water/ethanol (mol ratio=1:8:2) mixed solution at 80±2°C.; dialyse with distilled water for 3 days at room temperature to remove salts and ethanol in the solution; filtrate to remove insoluble impurities, so as to obtain an aqueous solution of silk fibroin; stir at 50-60 rpm and concentrate at 50±2° C. to obtain a silk fibroin solution with a concentration of 7-10 w/v %
B. Galactosylated chitosan (GC) preparation: Take 2.2 g chitosan and dissolve in an acetic acid-water solution with a concentration of 2.0 % and a volume of 30-40 mL; dilute the chitosan solution to a concentration of 4 w/v % with appropriate amount of TEMED/HCl buffer solution (pH4.7); add 0.14 g NHS, 0.6 g EDC and 2.2 g lactobionic acid (LA) respectively, and stirring react for 72 hours at room temperature; dialyse with distilled water for 4 days to obtain a galactosylated chitosan (GC) solution; evaporate partial water and concentrate to 4 w/v %.
C. Prepare a silk fibroin-galactosylated chitosan (SF/GC) mixed solution with a volume of 40 ml, wherein the mass ratio of silk fibroin to galactosylated chitosan is 6:4 and the final concentration of the solution is 4 w/v %; and precrosslink in a glutaraldehyde solution with a concentration of 0.5% and a volume of 4 ml for 15-30 minutes before dropping into an oil phase.
D. Put 160 ml liquid paraffin into a 500 ml beaker, add 6.4 ml Span 80, mix uniformly and stir at a constant speed of 250 rpm; drop the precrosslinked SF/GC mixed solution into the oil phase slowly, and stir for about 30 minutes at a constant speed of 250 rpm at room temperature, so as to obtain a white emulsion; drop a glutaraldehyde solution with a concentration of 2.5% and a volume of 3 ml into said white emulsion slowly, and stir until aqueous phase is crosslinking solidified.
E. Take 100 ml isopropanol, add a same volume of deionized water, and adjust the pH value to 9-10 by adding dilute NaOH solution.
F. Stir the solution obtained in step E at 250 rpm, add the white emulsion obtained in step D slowly, and keep stirring for 45 minutes; filtrate to obtain microspheres which do not stick to each other, and place the microspheres in a refrigeratory for 6 hours for solidifying.
G. Wash the microspheres with the diluted isopropanol solution obtained in step E, petroleum ether and deionized water, to remove the surface oil phase on the microspheres, and then sieve to obtain the microspheres with a diameter of 200-500 μm.
H. Immerse the microspheres obtained after sieving in a glycine solution with a concentration of 5 g/L for 3 hours, to remove unreacted glutaraldehyde, then wash with abundant distilled water; immerse the microspheres in a sucrose solution with a concentration of 40% for 3-5 minutes, then remove the excess sucrose solution; place the microspheres that has been immersd in sucrose solution into a refrigeratory at −20° C. freeze for 48 hours and then freeze dry for 48 hours, to obtain said macroporous microcarriers; then irradiate with cobalt 60-γ radial to sterilize said macroporous microcarriers after subpackage.

### The Second Embodiment

A. Silk fibroin preparation: Add 75 g raw silk to a sodium carbonate solution with a concentration of 5 g/L and a volume of 4 L, boil for 0.5 hour and repeat twice: wash with abundant distilled water to remove sericin protein and dry at 60-70° C. to obtain silk fibroin; dissolve appropriate amount of silk fibroin in a calcium chloride/water/ethanol (mol ratio=1:8:2) mixed solution at 80° C.; dialyse with distilled water for 3 days at room temperature to remove salts, ethanol and other small molecules in the solution; filtrate to remove insoluble impurities, so as to obtain an aqueous solution of silk fibroin; stir at 50-60 rpm and concentrate at 50±2° C. to obtain a silk fibroin solution with a concentration of 7-10 w/v %.
B. Galactosylated chitosan (GC) preparation: Take 1.1 g chitosan and dissolve in an acetic acid-water solution with a concentration of 2.0% and a volume of 15-20 mL; dilute the chitosan solution to a concentration of 4 w/v % with appropriate amount of TEMED/HCl buffer solution (pH4.7); add 0.07 g NHS, 0.3 g EDC and //1. ig lactobionic acid (LA) respectively, and stirring react for 72 hours at room temperature; dialyse with distilled water for 3 days to obtain a galactosylated chitosan (GC) solution; evaporate partial water and concentrate to 4 w/v %.
C. Prepare a silk fibroin-galactosylated chitosan-chitosan mixed solution with a volume of 40 ml, wherein the mass ratio of silk fibroin : galactosylated chitosan : chitosan is 3:1:1 and the final concentration of the solution is 4 w/v %; and stir uniformly.
D. Put 160 ml liquid paraffin into a 500 ml beaker, add 6.4 ml Span 80, mix uniformly and stir at a constant speed of 200 rpm; drop the silk fibroin-galactosylated chitosan-chitosan mixed solution into the oil phase slowly under stirring, and keep stirring for 30 minutes at a constant speed of 200 rpm at room temperature, so as to obtain a white emulsion; drop a glutaraldehyde solution with a concentration of 2.5% and a volume of 4ml into said white emulsion slowly, and stir for 3 hours until aqueous phase is crosslinking solidified.
E. Take 100 ml isopropanol, add a same volume of deionized water, and adjust the pH value to 9-10 by adding dilute NaOH solution.
F. Stir the diluted isopropanol solution obtained in step E at 300 rpm, add the white emulsion obtained in step D slowly and keep stirring for 45 minutes; filtrate to obtain microspheres which do not stick to each other, and place the microspheres in a refrigeratory at 4° C. for 6 hours for solidifying.
G. Wash the microspheres with the diluted isopropanol solution obtained in step E. petroleum ether, and deionized water, to remove the surface oil phase on the microspheres, and then sieve to obtain the microspheres with a diameter of 200-400 μm.
H. Immerse the microspheres obtained after sieving in a glycine solution with a concentration of 5 g/L for 2 hours, to remove unreacted glutaraldehyde, then wash with abundant distilled water; immerse the microspheres in a sucrose solution with a concentration of 40% for 3-5 minutes, then remove the excess sucrose solution; place the microspheres that has been immersd in sucrose solution into a refrigeratory at −20° C., freeze for 48 hours and then freeze dry for 48 hours, to obtain said macroporous microcarriers; then irradiate with cobalt 60-γ radial to sterilize said macroporous microcarriers after subpackage.

### The Third Embodiment

A. Silk fibroin preparation: Add 75 g raw silk to a sodium carbonate solution with a concentration of 5 g/L and a volume of 2 L, boil for 0.5 hour and repeat twice; wash with abundant distilled water to remove sericin protein and dry at 60-70° C. to obtain silk fibroin; dissolve appropriate amount of silk fibroin in a calcium chloride/water/ethanol (mol ratio=1:8:2) mixed solution at 80±2° C.; dialyse with distilled water for 3 days at room temperature to remove salts and ethanol in the solution; filtrate to remove insoluble impurities, so as to obtain an aqueous solution of silk fibroin; stir at 50-60 rpm and concentrate at 50±2° C. to obtain a silk fibroin solution with a concentration of 7-10 w/v %.
B. Galactosylated chitosan (GC) preparation: Take 2.2 g chitosan and dissolve in an acetic acid-water solution with a concentration of 2.0% and a volume of 30-40 mL; dilute the chitosan solution to a concentration of 4 w/v % with appropriate amount of TEMED/HCl buffer solution (pH4.7); add 0.14 g NHS, 0. 6g E1)C and 2.2 g lactobionic acid (LA) respectively, and stirring react for 72 hours at room temperature; dialyse with distilled water for 4 days to obtain a galactosylated chitosan (GC) solution; evaporate partial water and concentrate to 4 w/v %.
C. Prepare a silk fibroin-galactosylated chitosan-chitosan mixed solution with a volume of 30 ml, wherein the mass ratio of silk fibroin : galactosylated chitosan: chitosan is 5:3:2 and the final concentration of the solution is 5 w/v %; and stir uniformly
D. Put 150 ml liquid paraffin into a 500 ml beaker, add 6 ml Span 80, mix uniformly and stir at a constant speed of 250 rpm; drop the silk fibroin-galactosylated chitosan-chitosan mixed solution into the oil phase slowly under stirring, and keep stirring for 30 minutes at a constant speed of 250 rpm at room temperature, so as to obtain a white emulsion; drop a glutaraldehyde solution with a concentration of 2.5% and a volume of 3 ml into said white emulsion slowly, and stir for 3 hours until aqueous phase is crosslinking solidified.
E. Take 100 ml isopropanol, add a same volume of deionized water, and adjust the pH value to 9-10 by adding dilute NaOH solution.
F. Stir the diluted isopropanol solution obtained in step E at 300 rpm, add the white emulsion obtained in step 1) slowly, and keep stirring for 45 minutes; filtrate to obtain microspheres which do not stick to each other, and place the microspheres in a refrigeratory at 4° C. for 6 hours for solidifying.
G. Wash the microspheres with the diluted isopropanol solution obtained in step E, petroleum ether and deionized water, to remove the surface oil phase on the microspheres, and then sieve to obtain the microspheres with a diameter of 200-400 μm.
H. Immerse the microspheres obtained after sieving in a glycine solution with a concentration of 5 g/L for 4 hours, to remove unreacted glutaraldehyde, then wash with abundant distilled water; immerse the microspheres in a sucrose solution with a concentration of 40% for 3-5 minutes, then remove the excess sucrose solution; place the microspheres that has been immersd in sucrose solution into a refrigeratory at −20° C., freeze for 48 hours and then freeze dry for 48 hours, to obtain said macroporous microcarriers; then irradiate with cobalt 60-γ radial to sterilize said macroporous microcarriers after subpackage.

### The Fourth Embodiment

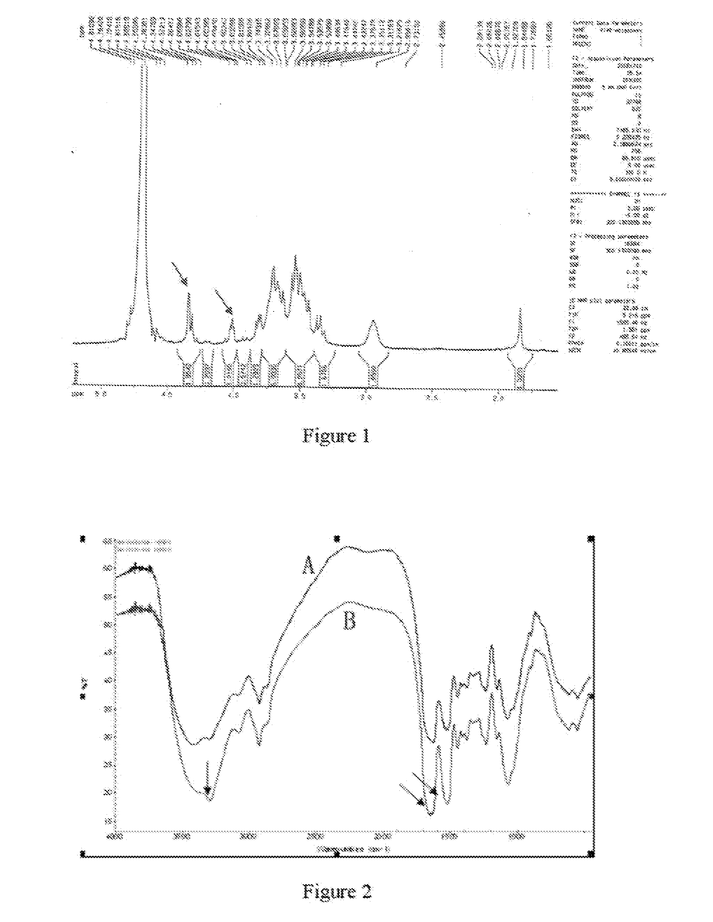

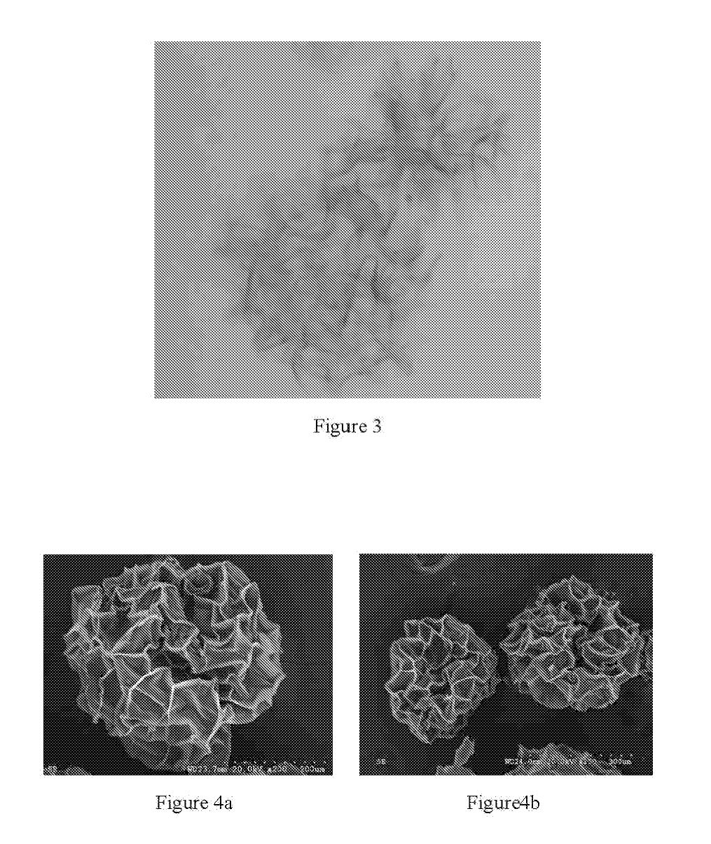

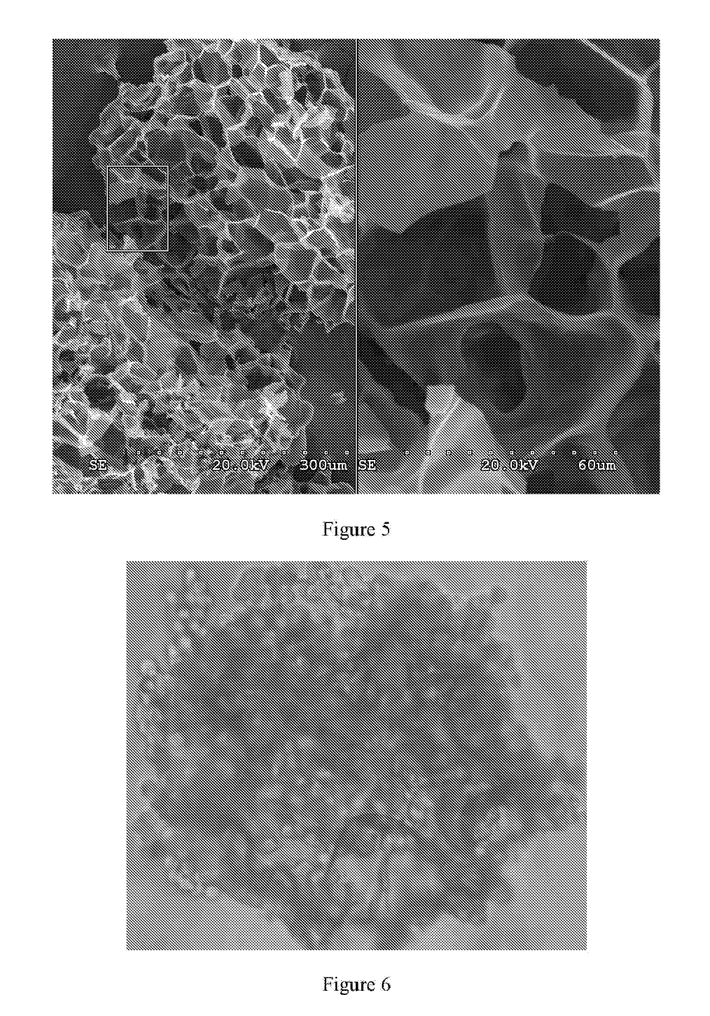

The macroporous microcarriers obtained by foresaid embodiments were analyzed by NMR and infra-red, and their ¹HNMR and FITR spectrums are shown in FIGS. 1 and 2. Said macroporous microcarriers were observed under inverted microscope and scanning electron microscope, and their images are shown in FIGS. 3-5. In the ¹HNMR spectrum of galactosylated chitosan (GC) shown in FIG. 1, the arrowheads indicate the characteristic peaks of galactosyl, and ¹HNMR assignments for GC are as follows: ¹HNMR (300 MHz, D₂O/F₃CCOOH) δ: 1.952 (H of —COCH₃), 4.755 (H1), 4.131, 4.415 (H1′, Hc), 3.3-4.0 (H3, H4, H5, H6, H2′, H3′, H4′, H5′, H6′, Ha, Hb, Hd, He), 3.066 (H₂), which prove that galactosyls have been covalent bound to the molecular chain of chitosan (CS) by chemical crosslinking. FIG. 2 show's the FITR spectrums of SF/CS (A) and SF/GC (B) macroporous microcarriers, wherein absorption peaks I and II of amide bonds (as shown by the right arrowheads) have chemical shifts and wider peak values, because they are formed by the ractions between the carboxyl groups of lactobionic acids and the amino groups of chitosan; the peak 1070.7 cm⁻¹ is an absorption peak of C—O stretching vibration of the galactosyl; and the absorption peak of OH group becomes wider and stronger (as shown by the left arrowhead), because the number of OH groups become larger after the galactosyls are grafted into the chitosan. FIG. 3 is a morphological image of the macroporous microcarriers under inverted microscope, wherein the macroporous microcarriers are white or light yellow semitransparent spheres and have uniform porous structure, and their three-dimensional structures could be seen under gently shaking. The porous structure of the macroporous microcarrier enlarges the specific surface area, provides a larger space for cell growth and increases observation convenience, thus the macroporous microcarrier is suitable for high density hepatocyte culture. FIGS. 4a and 4b are SEM images of the macroporous microcarriers, wherein the surface of the microcarrier has uniform porous structure with outward openings and trumpet shapes. Compared with the commercial macroporous microcarriers, the macroporous microcarrier of the present invention has larger apertures and outward openings, which would benefit cell adhesion and growth, and could provide enough pore space for the cells to enter the interior of the microcarrier. FIG. 5 shows an enlarged section view of the SF/GC macroporous microcarrier, wherein an interior porous structure can be seen and the pores are interconnected, which would benefit cell migration and growth inside the microcarrier and benefit internal substance exchange.

Wash the macroporous microcarriers of the present invention with PBS for 3 times after sterilization, then immerse in basic culture medium over night, and then add into a 48-well culture plate. Drop the CL-1 hepatocyte suspension into the culture plate and culture with the macroporous microcarriers. Change the medium every other day and observe the growth situation of the hepatocytes during the changing operation, including cell adhesion on the material, proliferation and morphological change. FIG. 6 shows an image of CL-1 cells with SF/GC macroporous microcarrier observed under inverted, microscope at the sixth day of the culture on the culture plate, wherein the cells locate, inside the macroporous microcanier, gather into multicellular clusters, form spherical growth and well adhere onto the microcarrier.

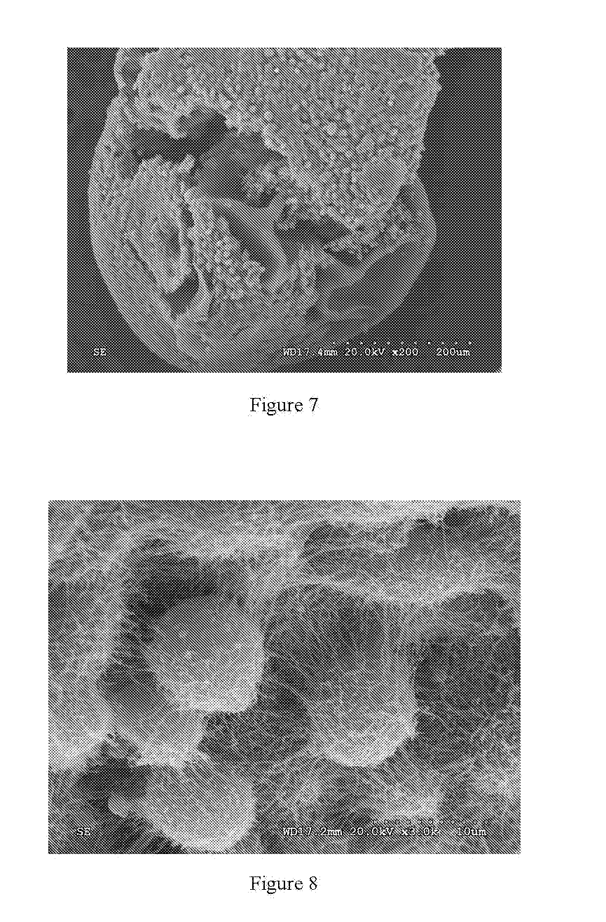

FIG. 7 shows an image of CL-1 cells with SF/GC macroporous microcarrier observed under scanning electron microscope (SEM) at the eighth day of the culture, wherein the cells are spherical, adhere onto the macroporous microcarrier, and gather into multicellular clusters. The cells strongly adhere onto the macroporous microcarrier because of the molecular recognition function between the ASGPR on the surface of the hepatocyte and the galactosyls on the microcarrier. Further, as the culture processes, the microcarriers would gather, the cell density would increase and the extracellular matrix would increase. FIG. 8 shows an enlarged image of CL-1 cells with SF/GC macroporous microcarrier observed under scanning electron microscope (SEM) at the eighth day of the culture, wherein the cells are spherical, have great microvilli, locate inside the macroporous microcarrier and gather for growth.

Application of the Macroporous Microcarrier

A method for large-scale hepatocyte culture using silk fibroin-galactosylated chitosan (SF/GC) macroporous microcarriers under microgravity rotary culture condition, comprises following steps:
I. SF/GC macroporous microcarriers pretreatment; Put desired amount of SF/GC macroporous microcarriers in a 50 ml plastic centrifuge tube, then add a phosphate buffer solution without calcium and magnesium (PBS) which has a concentration of 0.1 mol/L and a pH value of 7.0 for immersing over night, remove the PBS and wash with fresh PBS for 3 times, high-pressure sterilize at 121° C. for 30 minutes, remove the PBS and wash with culture medium for twice, and then immerse in serum-containing culture medium for above 10 hours.
II. Concentrated static cell inoculation: Dissociate and collect the hepatocyte suspension on a culture plate, add it to foresaid microcarriers according to desired inoculation density and mix gently; take out an aseptic microgravity rotary culture flask with large cross section under aseptic condition, open the flask cap and two pore valves, slowly inject the hepatocyte suspension with SF/GC macroporous microcarriers into the microgravity rotary culture flask by injector, and then add serum-containing culture medium up to 60% of the volume of the flask by another injector: take, out the two injectors, put back the cap and slightly loose; lay the culture flask in a CO, incubator statically under a temperature of 37° C. and a CO₂ concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors, one of which has serum-containing culture medium and the other one of which is empty, insert the injectors into two sampling pores respectively, open the sampling, pore valves, slowly inject the serum-containing culture medium of one injector into the flask, and draw out the air bubbles inside the flask by the empty injector, so as to assure that no air bubbles and eddy would be produced during the entire culture; after the flask is full of culture medium, scrub the flask opening and flask wall with 75% ethanol for 3 times, lay the culture flask in a CO₂ incubator and install it on a culture equipment. The rotary speed of the flask could be adjusted according to the amount of the microcarriers and the amount of the cells, as long as the microcarriers would not adhere on the flask wall during the rotation. In this experiment, the initial rotary speed is set at 7.6˜9 rpm.

### The Fifth Embodiment

Human hepatocyte (CL-1 cell) culture with silk fibroin-galactosylated chitosan (SF/GC) macroporous microcarriers under static or microgravity rotary culture condition
I. Complete medium preparation: Add 15 ml fetal bovine serum, 3.5 mmol HEPES, 10000U penicillin and 10000U streptomycin into every 100 ml DMEN medium (high glucose).
II. SF/GC macroporous microcarriers pretreatment: Put 0.1 g SF/GC macroporous microcarriers in a 50 ml plastic centrifuge tube, then add a phosphate buffer solution without calcium and magnesium (PBS) which has a concentration of 0.1 mol/L and a pH value of 7.0 for immersing over night, remove the PBS and wash with fresh PBS for 3 times high-pressure sterilize at 121° C. for 30 minutes, remove the PBS and wash with culture medium for twice, and then immerse in the complete medium obtained by step I for above 10 hours.
III. Cell Culture

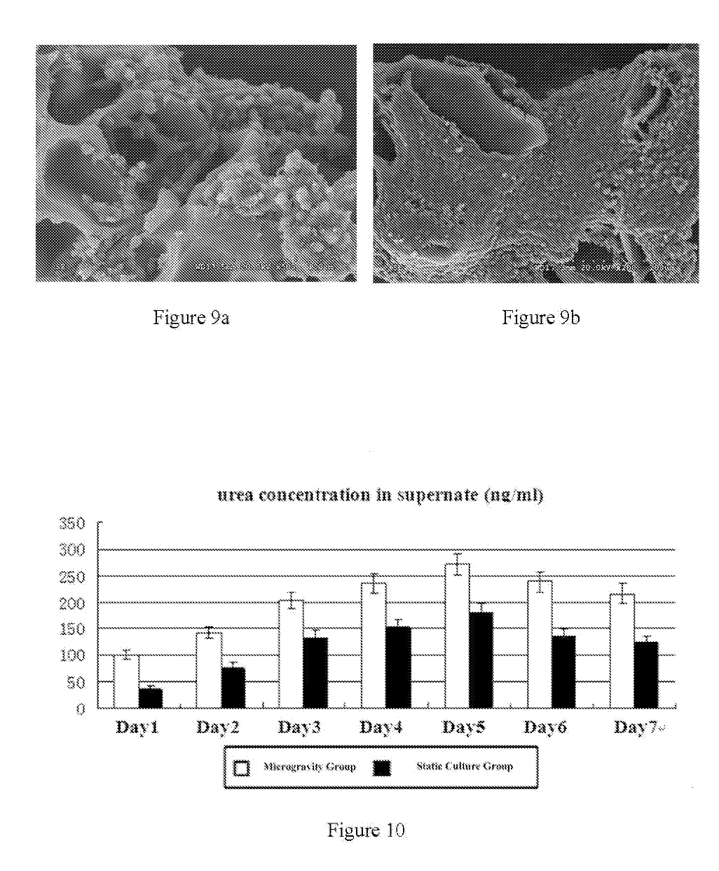

Microgravity rotary culture group (concentrated static cell inoculation): Dissociate and collect the human hepatocyte (CL-1 cell) suspension on a culture plate, add, the CL-1 cell suspension with a total amount of 2×10⁷ cells to foresaid microcarriers and mix gently; take out a 50 ml aseptic microgravity rotary culture flask with large cross section under aseptic condition, open the flask cap and two pore valves, slowly inject the hepatocyte suspension with SF/GC macroporous microcarriers into the microgravity rotary culture flask by injector, and then add the complete medium obtained by step I up to 30 ml by another injector; take out the two injectors, put back the cap and slightly loose; lay the culture flask in a CO₂ incubator statically under a temperature of 37° C. and a CO₂ concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors one of which has complete medium obtained by step I and the other one of which is empty, insert the injectors into two sampling pores respectively, open the sampling pore valves, slowly inject the medium of one injector into the flask, and draw out the air bubbles inside the flask by the empty injector, so as to assure that no air bubbles and eddy would be produced during the entire culture; after the flask is full of medium, scrub the flask opening and flask wall with 75% ethanol for 3 times, lay the culture flask in a CO₂ incubator and install it on a culture equipment. The rotary speed of the flask could be adjusted according to the amount of the microcarriers and the amount of the cells, as long as the microcarriers would not adhere on the flask wall during the rotation. In this experiment, the initial rotary speed is set at 7.6 rpm, the rotary speed would be adjusted once a day, and the medium changing amount is 30 ml a day.
ii. Static culture group (concentrated static cell inoculation): Dissociate and collect the human hepatocyte (CL-1 cell) suspension on a culture plate, add the CL-1 cell suspension with a total amount of 2×10⁷ cells to foresaid microcarriers and mix gently; take out a 50 ml aseptic microgravity rotary culture flask with large cross section under aseptic condition, open the flask cap and two pore valves, slowly inject the hepatocyte suspension with SF/GC macroporous microcarriers into the microgravity rotary culture flask by injector, and then add the complete medium obtained by step I up to 30 ml by another injector; take out the two injectors, put back the cap and slightly loose; lay the culture flask in a CO₂ incubator statically under a temperature of 37° C. and a CO₂ concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors, one of which has complete medium obtained by step I and the other one of which is empty insert the injectors into two sampling pores respectively, open the sampling pore valves, slowly inject the medium of one injector into the flask, and draw out the air bubbles inside the flask by the empty injector, so as to assure that no air bubbles and eddy would be produced during the entire culture; after the flask is full of medium, scrub the flask, opening and flask wall with 75% ethanol for 3 times, lay the culture flask in a CO₂ incubator and the medium changing amount is 30 ml a day.
IV. Biofunction test: Collect the supernate as sample at daily medium changing, centrifugate the supernate at 2000 r/min for 10 minutes and test the urea content by Beckman automatic biochemistry analyzer.
V. Scanning electron microscope observation: Wash the sample with PBS for 3 times fix by 2% glutaraldehyde for 0.5 hour and then fix by 1% osmic acid for 0.5 hour, dehydrate with gradient ethanol, process in isoamyl acetate for above 4 hours, dry by critical evaporator and then sputter with platinum ion under vacuum condition. The cell growth situation is observed and shot by Japan S450 scanning electron microscope. Test results: Microgavity SF/GC macroporous microcarriers were used for the large-scale extracorporeal hepatocyte culture in the present invention, compared with the traditional static three-dimensional culture, it is more conducive to hepatocyte proliferation, differentiation and intercellular contaction, and a compact three-dimensional structural tissue would be formed to achieve large-scale and high-density extracorporeal hepatocyte culture, and to improve hepatocyte functions (as shown in FIGS. 9a, 9b and 10).

### The Sixth Embodiment

Human hepatocyte (CL-1 cell) culture with microcarriers under microgravity rotary culture condition (silk fibroin-galactosylated chitosan (SF/GC) macroporous microcarriers, solid microcarriers: cytodex 3)
I. Complete medium preparation: Add 15 ml fetal bovine serum, 3.5 mmol HEPES, 10000U penicillin and 10000U streptomycin into every 100 ml DMEN medium (high glucose).
II. Microcarriers Pretreatment
i. SF/GC macroporous microcarriers pretreatment: Put 0.2 g SF/GC macroporous microcarriers in a 50 ml plastic centrifuge tube, then add a phosphate buffer solution without calcium and magnesium (PBS) which has a concentration of 0.1 mol/L and a pH value of 7.0 for immersing over night, remove the PBS and wash with fresh PBS for 3 times, high-pressure sterilize at 121° C. for 30 minutes, remove the PBS and wash with culture medium for twice, and then immerse in the complete medium obtained by step I for above 10 hours.
ii. Solid microcarriers (cytodex 3) pretreatment: Put 0.25 g solid microcarriers (cytodex 3) in a 50 ml plastic centrifuge tube, then add a phosphate buffer solution without calcium and magnesium (PBS) which has a concentration of 0.1 mol/l, and a pH value of 7.0 for immersing over night remove the PBS and wash with fresh PBS for 3 times, high-pressure sterilize at 121° C. for 30 minutes, remove the PBS and wash with culture medium for twice, and then immerse in the complete medium obtained by step I for above 10 hours.
III. Human hepatocyte (CL-1 cell) culture with microcarriers under microgravity rotary culture condition

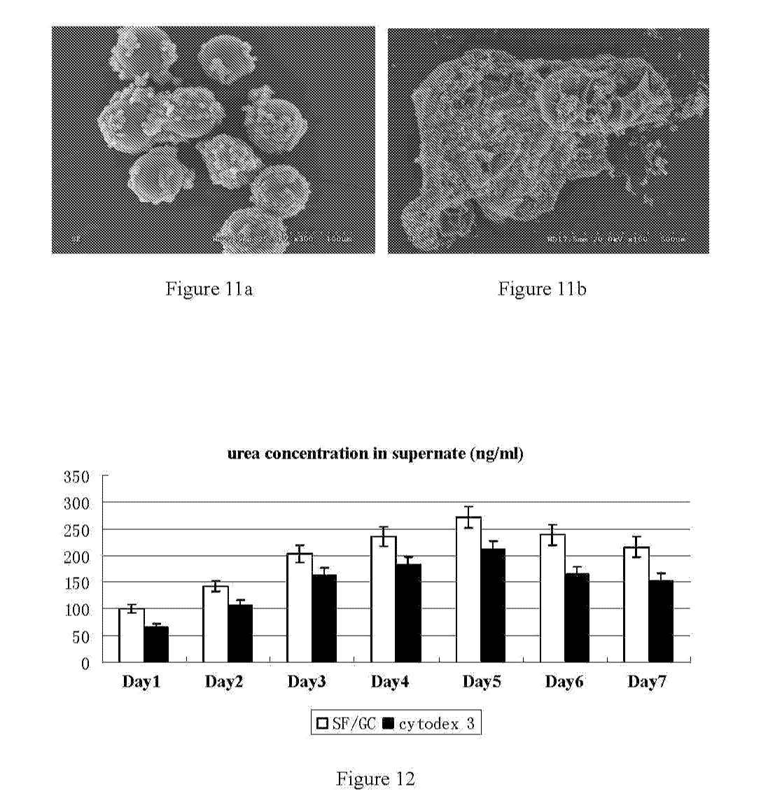

Human hepatocyte (CL-1 cell) culture with microgravity SF/GC macroporous microcarriers: Dissociate and collect the human, hepatocyte (CL-1 cell) suspension on a culture plate, add the CL-1 cell suspension with a total amount of 2×10⁷ cells to foresaid microcarriers and mix gently; take out a 50 ml aseptic microgravity rotary culture flask with large cross section under aseptic condition, open the flask cap and two pore valves, slowly inject the hepatocyte suspension with SF/GC macroporous microcarriers into the microgravity rotary culture flask by injector, and then add the complete medium obtained by step I up to 30 ml by another injector; take out the two injectors, put back the cap and slightly loose; lay the culture flask in a CO₂ incubator statically under a temperature of 37° C. and a CO₂ concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors, one of which has complete medium obtained by step I and the other one of which is empty insert the injectors into two sampling pores respectively, open the sampling pore valves, slowly inject the medium of one injector into the flask, and draw out the air bubbles inside the flask by the empty injector so as to assure that no air bubbles and eddy would be produced during the entire culture; after the flask is full of medium, scrub the flask opening and flask wall with 75% ethanol for 3 times, lay the culture flask in a CO₂ incubator and install it on a culture equipment. The rotary speed of the flask could be adjusted according to the amount of the microcarriers and the amount of the cells, as long as the microcarriers would not adhere on the flask wall during the rotation. In this experiment, the initial rotary speed is set at 7.6 rpm, the rotary speed would be adjusted once a day, and the medium changing amount is 30 ml a day.
ii. Human hepatocyte (CL-1 cell) culture with microgravity solid microcarriers (cytodex 3): Dissociate and collect the human hepatocyte (CL-1 cell) suspension on a culture plate, add the CL-1 cell suspension with a total amount of 2×10⁷ cells to foresaid microcarriers and mix gently; take out a 50 ml aseptic microgravity rotary culture flask with large cross section under aseptic condition, open the flask cap and two pore valves, slowly inject the hepatocyte suspension with cytodex 3 microcarriers into the microgravity rotary culture flask by injector, and then add the complete medium obtained by step I up to 30 ml by another injector; take out the two injectors put back the cap and slightly loose; lay the culture flask in a CO₂ incubator statically under a temperature of 37° C. and a CO₂ concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors, one of which has complete medium obtained by step I and the other one of which is empty, insert the injectors into two sampling pores respectively, open the sampling pore valves, slowly inject the medium of one injector into the flask, and draw out the air bubbles inside the flask by the empty injector so as to assure that no air bubbles and eddy would be produced during the entire culture; after the flask is full of medium scrub the flask opening and flask wall with 75% ethanol for 3 times, lay the culture flask in a CO₂ incubator and install it on a culture equipment. The rotary speed of the flask could be adjusted according to the amount of the microcarriers and the amount of the cells, as long as the microcarriers would not adhere on the flask wall during the rotation. In this experiment, the initial rotary speed is set at 7.6 rpm, the rotary speed would be adjusted once a day, and the medium changing amount is 30 ml a day.
IV. Biofunction test: Collect the supernate as sample at daily medium changing, centrifugate the supernate at 2000 r/min for 10 minutes and test the urea content by Beckman automatic biochemistry analyzer.
V. Scanning electron microscope observation: Wash the sample with PBS for 3 times fix by 2% glutaraldehyde for 0.5 hour and then fix by 1% osmic acid for 0.5 hour dehydrate with gradient ethanol, process in isoamyl acetate for above 4 hours, dry by critical evaporator and then sputter with platinum ion under vacuum condition. The cell growth situation is observed and shot by Japan S450 scanning electron microscope. Test results: SF/GC macroporous microcarriers were used as scaffold material for the large-scale extracorporeal hepatocyte culture in the present invention, compared with the traditional solid scaffold material (cytodex 3), it has larger surface area/volume ratio and a sinus gap structure extremely similar with in-vivo liver sinus structure, it is more conducive to intercellular contaction, oxygen and nutrient components transportation and metabolic products excretion, so as to further improve the culture density and the cell functions of hepatocytes (as shown in FIGS. 11a, 11b and 12).

### The Seventh Embodiment

Human hepatocyte (CL-1 cell) culture with microcarriers under microgravity rotary culture condition (silk fibroin-galactosylated chitosan (SF/GC) macroporous microcarriers porous micro carriers: cytopore)
I. Complete medium preparation: Add 15 ml fetal bovine serum. 3.5 mmol HEPES, 10000U penicillin and 10000U streptomycin into every 100 ml DMEN medium (high, glucose).
II. Microcarriers Pretreatment
i. SF/GC macroporous microcarriers pretreatment: Put 0.2 g SF/GC macroporous microcarriers in a 50 ml plastic centrifuge tube, then add a phosphate buffer solution without calcium and magnesium (PBS) which has a concentration of 0.1 mol/L and a pH value of 7.0 for immersing over night, remove the PBS and wash with fresh PBS for 3 times, high-pressure sterilize at 121° C. for 30 minutes, remove the PBS and wash with culture medium for twice, and then immerse in the complete medium obtained by step I for above 10 hours.
ii. Porous microcarriers (cytopore) pretreatment: Put 0.2 g porous microcarriers (cytopore) in a 50 ml plastic centrifuge tube, then add a phosphate buffer solution without calcium and //magnesium (PBS) which has a concentration of 0.1 mol/L and a pH value of 7.0 for immersing over night, remove the PBS and wash with fresh PBS for 3 times, high-pressure sterilize at 121° C. for 30 minutes, remove the PBS and wash with culture medium for twice, and then immerse in the complete medium obtained by step I for above 10 hours.
III. Human hepatocyte (CL-1 cell) culture with microcarriers under microgravity rotary culture condition
i. Human hepatocyte (CL-1 cell) culture with microgravity SF/GC macroporous microcarriers: Dissociate and collect the human hepatocyte (CL-1 cell) suspension on a culture plate, add the CL-1 cell suspension with a total amount of 2×10⁷ cells to foresaid microcarriers and mix gently; take out a 50 ml aseptic microgravity rotary culture flask with large cross section under aseptic condition, open the flask cap and two pore valves, slowly inject the hepatocyte suspension with SF/GC macroporous microcarriers into the microgravity rotary culture flask by injector, and then add the complete medium obtained by step I up to 30 ml by another injector; take out the two injectors, put back the cap and slightly loose: lay the culture flask in a CO₂ incubator statically under a temperature of 37° C. and a CO₂ concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors, one of which has complete medium obtained by step I and the other one of which is empty, insert the injectors into two sampling pores respectively open the sampling pore valves, slowly inject the medium of one injector into the flask, and draw out the air bubbles inside the flask by the empty injector, so as to assure that no air bubbles and eddy would be produced during the entire culture; after the flask is full of medium, scrub the flask opening and flask wall with 75% ethanol for 3 times, lay the culture flask in a CO₂ incubator and install it on a culture equipment. The rotary speed of the flask could be adjusted according to the amount of the microcarriers and the amount of the cells, as long as the microcarriers would not adhere on the flask wall during the rotation. In this experiment, the initial rotary speed is set at 7.6 rpm, the rotary speed would be adjusted once a day, and the medium changing amount is 30 ml a day.

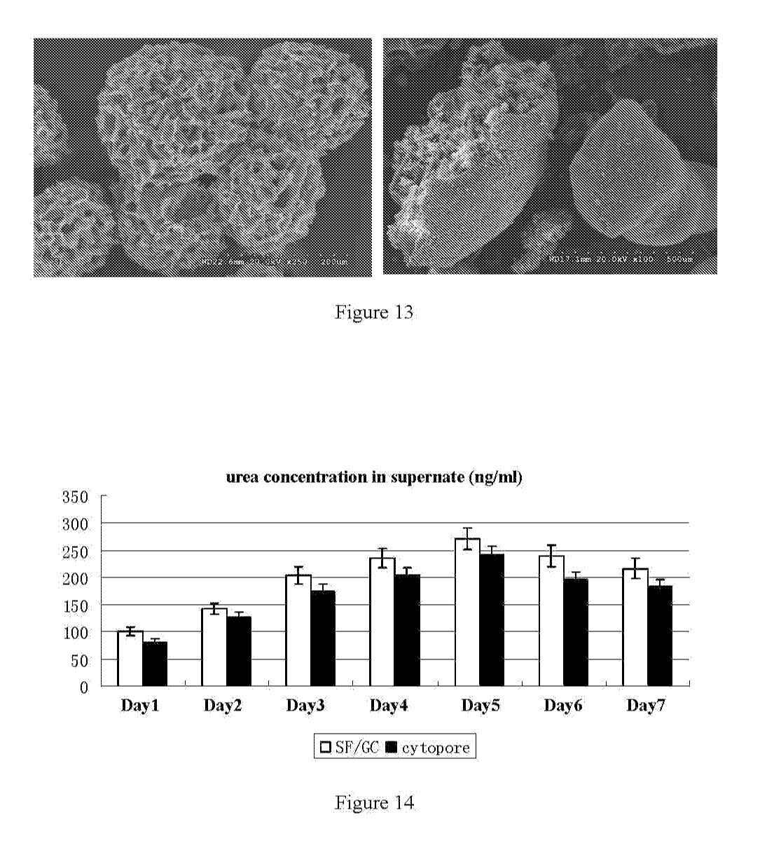

Human hepatocyte (CL-1 cell) culture with microgravity porous microcarriers (cytopore): Dissociate and collect the human hepatocyte (CL-1 cell) suspension on a culture plate add the CL-1 cell suspension with a total amount of 2×10⁷ cells to foresaid microcarriers and mix gently; take out a 50 ml aseptic microgravity rotary culture flask with large cross section under aseptic condition, open, the flask cap and two pore valves, slowly inject the hepatocyte suspension with porous microcarriers (cytopore) into the microgravity rotary culture flask by injector, and then add the complete medium obtained by step I up to 30 ml by another injector; take out the two injectors put back the cap and slightly loose; lay the culture flask in a CO₂ incubator statically under a temperature of 37° C. and a CO, concentration of 5%; take out the culture flask every 8 hours and shake slightly for 1 minute; after 24 hours, take out the culture flask, place on a super clean bench which has been sterilized by ultraviolet ray, and scrub the flask cap and flask wall with 75% ethanol for 3 times; open the flask cap, take two 10 ml aseptic injectors, one of which has complete medium obtained by step I and the other one of which is empty, insert the injectors into two sampling pores respectively, open the sampling pore valves, slowly inject the medium of one injector into the flask, and draw out the air bubbles inside the flask by the empty injector, so as to assure that no air bubbles and eddy would be produced during the entire culture; after the flask is full of medium, scrub the flask opening and flask wall with 75% ethanol for 3 times, lay the culture flask in a CO₂ incubator and install it on a culture equipment. The rotary speed of the flask could be adjusted, according to the amount of the microcarriers and the amount of the cells, as long as the microcarriers would not adhere on the flask wall during the rotation. In this experiment the initial rotary speed is set at 7.6 rpm the rotary speed would be adjusted once a day, and the medium changing amount is 30 ml a day.
IV. Biofunction test: Collect the supernate as sample at daily medium changing centrifugate the supernate at 2000 r/min for 10 minutes and test the urea content by Beckman automatic biochemistry analyzer.
V. Scanning electron microscope observation: Wash the sample with PBS for 3 times, fix by 2% glutaraldehyde for 0.5 hour and then fix by 1% osmic acid for 0.5 hour, dehydrate with gradient ethanol, process in isoamyl acetate for above 4 hours, dry by critical evaporator and then sputter with platinum ion under vacuum condition. The cell growth situation is observed and shot by Japan S450 scanning electron microscope. Test results: Galactosyl is a ligand specific to asialoglycoprotein receptor on the surface of hepatocyte, the SF/GC macroporous microcarriers used in the present invention were modified by galactosyls under the activation of EDC and NHS; compared with the traditional porous microcarriers (cytopore), it is more conducive to hepatocyte adhesion on scaffold materials, and it can further improve the culture density and the cell functions of hepatocytes (as shown in FIGS. 13a, 13b and 14).

It is to be understood that the present invention includes but is not limited to the disclosed embodiments. The scopes of the appended claims encompass all the modifications and the equivalents which are apparent to those skilled in the art.

1-17. (canceled)

18. A macroporous microcarrier, which is a sphere prepared from silk fibroin and galactosylated chitosan under an effect of crosslinker.

19. A macroporous microcarrier according to claim 18, wherein based on a total weight of the sphere, a content of silk fibroin is 50-80 wt % and a content of galactosylated chitosan is 15-40 wt %.

20. A macroporous microcarrier according to claim 18, wherein said crosslinker is glutaraldehyde.

21. A macroporous microcarrier according to claim 18, wherein a diameter of said microcarrier is 200-500 μm, and an aperture of said microcarrier is 40-80 μm.

22. A preparation method for the macroporous microcarrier of claim 18, comprising steps of:
A. mixing silk fibroin solution and galactosylated chitosan solution according to a ratio to obtain a silk fibroin-galactosylated chitosan mixed solution;
B. dropping said silk fibroin-galactosylated chitosan mixed solution into an oil phase of emulsifier under stirring, to obtain a white emulsion; dropping crosslinker into said white emulsion slowly, and stirring until aqueous phase is crosslinking solidified;
C. adding said white emulsion obtained by step B into a polar solvent under stirring, keepping stirring until microspheres are formed, and then filtrating to obtain microspheres which do not stick to each other;
D. removing the oil phase on the surface of said microspheres by organic solvent,and sieving to obtain microspheres; and
E. removing the crosslinker residual in said microspheres, and freeze drying to obtain said macroporous microcarrier.

23. A preparation method according to claim 22, wherein said silk fibroin-galactosylated chitosan mixed solution has a final concentration of 4-7 w/v %.

24. A preparation method according to claim 22, wherein said emulsifier comprises paraffin and water-in-oil emulsifier.

25. A preparation method according to claim 22, wherein said crosslinker is glutaraldehyde.

26. A preparation method according to claim 22, wherein said polar solvent selects from one or more of a group of isopropanol, ethanol and acetone; and said polar solvent has a pH value of 9-10.

27. A preparation method according to claim 22, wherein said organic solvent is dilute isopropanol and/or petroleum ether.

28. A preparation method according to claim 22, wherein said step E comprises a process of immersing said microspheres in a sucrose solution before freeze drying.

29. A preparation method according to claim 22, wherein said preparation method comprises a process of sterilization after step E.

30. A preparation method according to claim 29, wherein said sterilization process is irradiating with cobalt 60-γ radial or autoclaving.

31. A method for large-scale extracorporeal hepatocyte culture comprising steps of:
I. providing said macroporous microcarriers of claim 1; and
II. applying said macroporous microcarriers to microgravity rotary culture system.

32. A method according to claim 31, wherein said method comprises a process of immersing said macroporous microcarriers in a phosphate buffer solution without calcium and magnesium which has a concentration of 0.1 mol/L and a pH value of 7.0 over night, and then immersing in serum-containing culture medium for at least 10 hours before step II.

33. A method according to claim 31, wherein concentrated static inoculation method is used in said microgravity rotary culture system for cell inoculation, and a cell inoculation concentration is 2×10⁵/ml to 1×10⁶/ml.

34. A method according to claim 33, wherein an initial medium volume of the concentrated static inoculation method is 40%˜90% of a volume of culture flask.

35. A method according to claim 33, wherein a static time used in the concentrated static inoculation method is 12˜24 hours.

36. A method according to claim 33, wherein an initial rotary speed used in the concentrated static inoculation method is 7.6˜9 rmp.

37. A method according to claim 33, wherein a culture medium used in the concentrated static inoculation method is MIEN or PRMI 1640; said culture medium contains 10%˜15% serum and contains HEPES with a concentration of 20 mmol/L˜50 mmol/L.
